(19) United States
(12) Patent Application Publication
(10) Pub. No.: US 2007/0060784 A1
(43) Pub. Date: Mar. 15, 2007

(54) System and method for rating severity of psoriasis for target area

(75) Inventor: Charles N. Ellis, Ann Arbor, MI (US)

Correspondence Address: CALFEE HALTER & GRISWOLD, LLP, 800 SUPERIOR AVENUE, SUITE 1400, CLEVELAND, OH, 44114, US

(73) Assignee: DATAcquire, Inc.

(21) Appl. No.: 11/222,518

(22) Filed: Sep. 9, 2005

Related U.S. Application Data
(63) Continuation-in-part of application No. 10/530,033, filed on Oct. 14, 2005.

(51) Int. Cl. A61N 5/00

(52) U.S. Cl. 600/1

(57) ABSTRACT

A system and method for rating the severity of psoriasis in a patient is provided. In one embodiment, the system includes: a first logic for categorizing psoriasis plaque qualities into one of a plurality of categories of increasing severity for at least one target area of the patient based on an assessment of each psoriasis plaque quality for each corresponding target area, the psoriasis plaque qualities including elevation plaque quality, erythema plaque quality, and scale plaque quality and a second logic for classifying the severity of psoriasis for each target area into one of a plurality of classes of increasing severity based on the categorized psoriasis plaque quality with priority given to the categorized elevation plaque quality over other categorized plaque qualities. In another embodiment, the system also includes a third logic for assessing the psoriasis plaque qualities of each target area of the patient.

This application is a continuation-in-part (CIP) of U.S. patent application Ser. No. 10/530,033 which is a U.S. national stage (35 U.S.C. § 371(c)) application from PCT Pat. App. No. PCT/US2003/033218, filed Oct. 20, 2003, the contents of which are fully incorporated herein by reference. U.S. patent application Ser. No. 10/530,033 and PCT Pat. App. No. PCT/US2003/033218 claim the benefit of U.S. Prov. Pat. App. Ser. No. 60/419,652, filed Oct. 18, 2002, the contents of which are fully incorporated herein by reference.

### BACKGROUND

The present invention relates to methods of rating the severity of psoriasis in a subject, more particularly a human patient.

Psoriasis is a debilitating autoimmune dermatological disease that affects about 2.6% of the population. Plaque psoriasis, the most common form of the disease, is characterized by red skin covered with silvery scales. Histologically the picture is one of disordered differentiation and hyperproliferation of keratinocytes within the psoriatic plaque with inflammatory cell infiltrates.

Onset occurs most often in early adult life, but also may begin in childhood or in aged people. Severity of the disease varies and is usually characterized by alternating periods of remission and flare-up. In more serious cases, psoriasis can affect up to 90% of the skin and can be life threatening.

New therapeutic agents are currently being developed to treat this skin disorder. Accurate, reliable, and appropriate documentation of the extent and severity of psoriasis is important in clinical practice, and essential for clinical trial research. In clinical trials, the objective quantification of the disease is critical to measure the efficacy of an investigational treatment by comparing the severity of disease before therapy to that measured after treatment. For a new therapy to achieve regulatory approval for marketing, its efficacy must be documented in clinical trials. Valid and reliable outcome measures are also important in evidence-based medicine to provide comparisons among similarly designed trials in the literature. Although outcome measures such as quality-of-life assessments are important methods of assessing patient morbidity and disease severity, the clinical evaluation of psoriasis severity currently is the primary efficacy variable in clinical trial research.

Despite the importance of psoriasis evaluation, there is a lack of consensus on the most appropriate measure.¹⁻⁴ The measures most often employed in clinical trials to measure psoriasis disease severity are the Psoriasis Area and Severity Index (PASI) and the Psoriasis Global Assessment (PGA). PASI was developed in 1978 by Fredriksson and Pettersson⁵ for use in a single clinical trial. Subsequently, the PASI became popular as a research tool, but is not used in clinical practice. The PASI results in a single score for psoriasis severity from zero to 72; the plaque qualities of erythema, infiltration, desquamation are weighted equally to each other in calculating the final score. The method for calculating such score is depicted in Table 1 below.

Table 1. Method for Calculating the Psoriasis Area and Severity Index (PAST)

The original description of the PASI involves the assessment over four body regions (head, trunk, upper and lower extremities) of infiltration, desquamation, and erythema, and body surface area involvement. The assessment for infiltration, desquamation, and erythema use a five-point scale:

| | |
|---|---|
| No symptoms | 0 |
| Slight | 1 |
| Moderate | 2 |
| Marked | 3 |
| Very marked | 4 |

Based on the extent of lesions in each anatomic region, the area affected is assigned a numerical value of one to 6:

| | |
|---|---|
| <10% of the body surface involved (per region) | 1 |
| 10-29% | 2 |
| 30-49% | 3 |
| 50-69% | 4 |
| 70-89% | 5 |
| 90-100% | 6 |

Since the head, upper extremities, trunk, and lower extremities, correspond to approximately 10, 20, 30, and 40% of body surface area, respectively, the PASI score can be calculated using the formula:
PASI=0.1(Eₕ+Iₕ+Dₕ)Aₕ+0.2(Eᵤ+Iᵤ+Dᵤ)Aᵤ+0.3(Eₜ+Iₜ+Dₜ)Aₜ+0.4(Eₗ+Iₗ+Dₗ)Aₗ
where E, I, D, and A denote the point score for erythema, infiltration, desquamation, and area, respectively, and h, u, t, and l denote head, upper extremities, trunk, and lower extremities, respectively. PASI varies in steps 0.1 units from 0.0 to 72.0, with the highest score representing near or complete body involvement with the most severe erythema, infiltration, and desquamation possible.

The PGA allows the investigator to assign a single estimate of the patient's overall severity of disease; typically, a seven-point scale from clear to severe is used, although many variations have been employed. The PGA evaluates disease severity in a more intuitive and clinically meaningful way than does the zero to 72 score of the PASI. In most versions of the PGA, the individual elements of psoriasis plaque morphology or degree of body surface area involvement are not quantified. A detailed description of one PGA method is depicted in Table 2 below.

TABLE 2
Description of a Physician's Global Assessment (PGA)

| | |
|---|---|
| Severe | Very marked plaque elevation, scaling, and/or |
| | erythema |
| Moderate to severe | Marked plaque elevation, scaling, and/or erythema |
| Moderate | Moderate plaque elevation, scaling, and/or erythema |
| Mild to moderate | Intermediate between moderate and mild |
| Mild | Slight scaling plaque elevation, scaling, and/or |
| | erythema |
| Almost clear | Intermediate between mild and clear |
| Clear | No signs of psoriasis (post-inflammatory |
| | hyperpigmentation may be present) |

The reliability of current clinical outcome measures is uncertain. Accordingly, new methods for rating the severity of psoriasis are needed.

### SUMMARY OF THE INVENTION

In one aspect, a system for rating the severity of psoriasis in a patient is provided. In one embodiment, the system includes: a first logic for categorizing psoriasis plaque qualities into one of a plurality of categories of increasing severity for at least one target area of the patient based on an -assessment of each psoriasis plaque quality for each corresponding target area, the psoriasis plaque qualities including elevation plaque quality, erythema plaque quality, and scale plaque quality and a second logic for classifying the severity of psoriasis for each target area into one of a plurality of classes of increasing severity based on the categorized psoriasis plaque quality with priority given to the categorized elevation plaque quality over other categorized plaque qualities.

In another aspect, a method for rating the severity of psoriasis of a patient is provided. In one embodiment, the method includes: a) assessing psoriasis plaque qualities for at least one target area of the patient, the psoriasis plaque qualities including elevation plaque quality, erythema plaque quality, and scale plaque quality, b) categorizing each assessed psoriasis plaque quality into one of a plurality of categories of increasing severity based on the corresponding assessed psoriasis plaque quality, and c) classifying the severity of psoriasis for each target area into one of a plurality of classes of increasing severity based on the categorized psoriasis plaque quality with priority given to the categorized elevation plaque quality over other categorized plaque qualities.

In another embodiment, the system for rating the severity of psoriasis in a patient includes: means for categorizing psoriasis plaque qualities into one of a plurality of categories of increasing severity for at least one target area of the patient based on an assessment of each psoriasis plaque quality for each corresponding target area, the psoriasis plaque qualities including elevation plaque quality, erythema plaque quality, and scale plaque quality, and means for classifying the severity of psoriasis for each target area into one of a plurality of classes of increasing severity based on the categorized psoriasis plaque quality with priority given to the categorized elevation plaque quality over other categorized plaque qualities.

The systems and methods provided herein are useful for evaluating the effects of therapeutic agents on psoriasis. The systems and methods are also useful for rating psoriasis to qualify patients for certain therapies (e.g., for insurance coverage or risk-benefit determinations).

In certain embodiments, the systems and methods also relate to a computer readable medium.

### BRIEF DESCRIPTION OF THE FIGURES

FIG. 1 shows a process for recording scores in a Lattice System Global Psoriasis Score-(LS-GPS) with four plaque quality categories.

FIG. 2 is a graphic representation of an exemplary embodiment of a system and method for a Lattice System Global Psoriasis Score (LS-GPS-1).

FIG. 3 is a graphic representation of another exemplary embodiment of a system and method for a Lattice System Global Psoriasis Score (LS-GPS-2).

FIG. 4 shows a process for recording scores in a Lattice System Target Psoriasis Score (LS-TPS) with four plaque quality categories.

FIG. 5 is a graphic representation of an exemplary embodiment of a system and method for an LS-TPS.

### DETAILED DESCRIPTION OF THE INVENTION

“Elevation” as used herein includes, but is not limited to, the visually observable, palpable, or measured overall height of the plaques on the subject above normal skin. The edge characteristics of the plaques may also be used to assess the extent of elevation of the plaques.

“Erythema” as used herein includes, but is not limited to, redness, or degree or quality of red color of the plaques of psoriasis or skin as measured or visually observed.

“Scale” as used herein includes, but is not limited to, the amount of stratum corneum that is present on the surface of the plaques of psoriasis or skin as measured, palpated, or visually observed.

Systems and methods for rating the severity of psoriasis are provided. In one embodiment, such methods comprise determining a global score of the severity of the disease. In one part, the score is based on the percentage of body surface showing signs of psoriasis. Such signs include increased elevation, erythema, and scale. Although it is possible to examine the head, neck, chest, back, abdomen, groin, buttocks, and both arms and both legs (including hands and feet) of the patient separately or in groupings such as the trunk (chest, back, and abdomen), it is preferred that the entire body be examined to assess such percentage. The global score is also based on the overall elevation, overall erythema, and overall scale of the psoriasis plaques in all the involved areas. The overall elevation of the plaques in the involved areas is given added weight in determining the global score, and, preferably, the overall level of scale is given a lesser weight in the determination. Preferably, overall elevation, erythema, and scale are divided into a plurality of categories ranging from zero or none up to the highest category. One example of a suitable designation for the category assessment is in four steps, namely none, mild, moderate, and marked. However, other designations may be used. Preferably the percentage of body showing signs of psoriasis is also divided into a plurality of ranges, such as, for example, 1-3%, 4-9%, 10-20%, 21-29%, 30-50%, and greater than 50%. However, other ranges may be used.

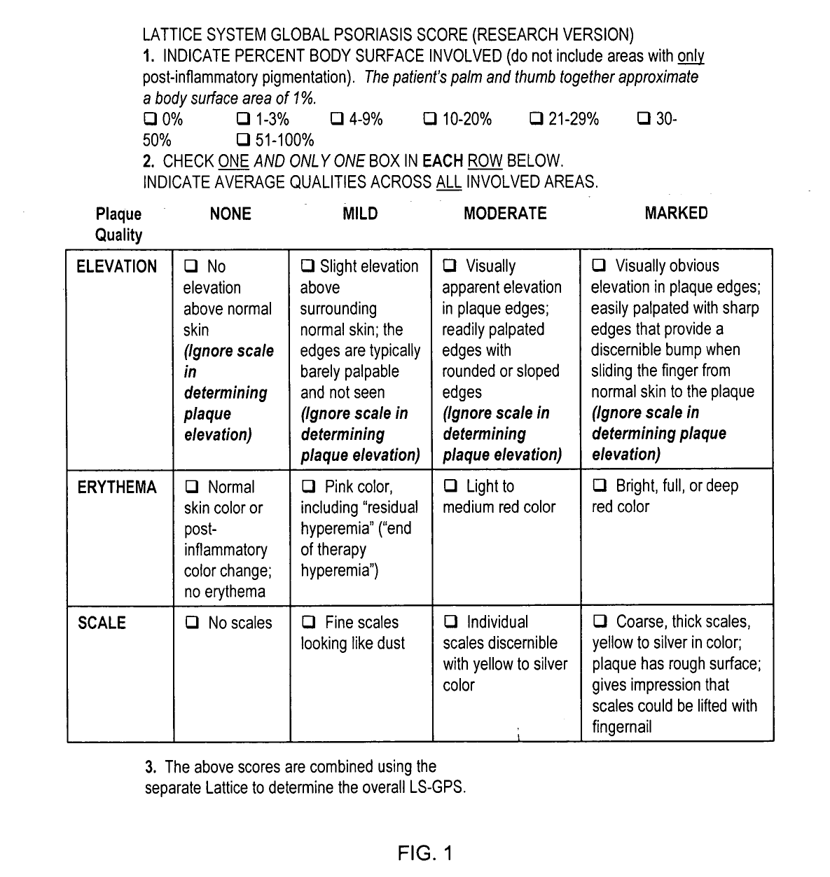

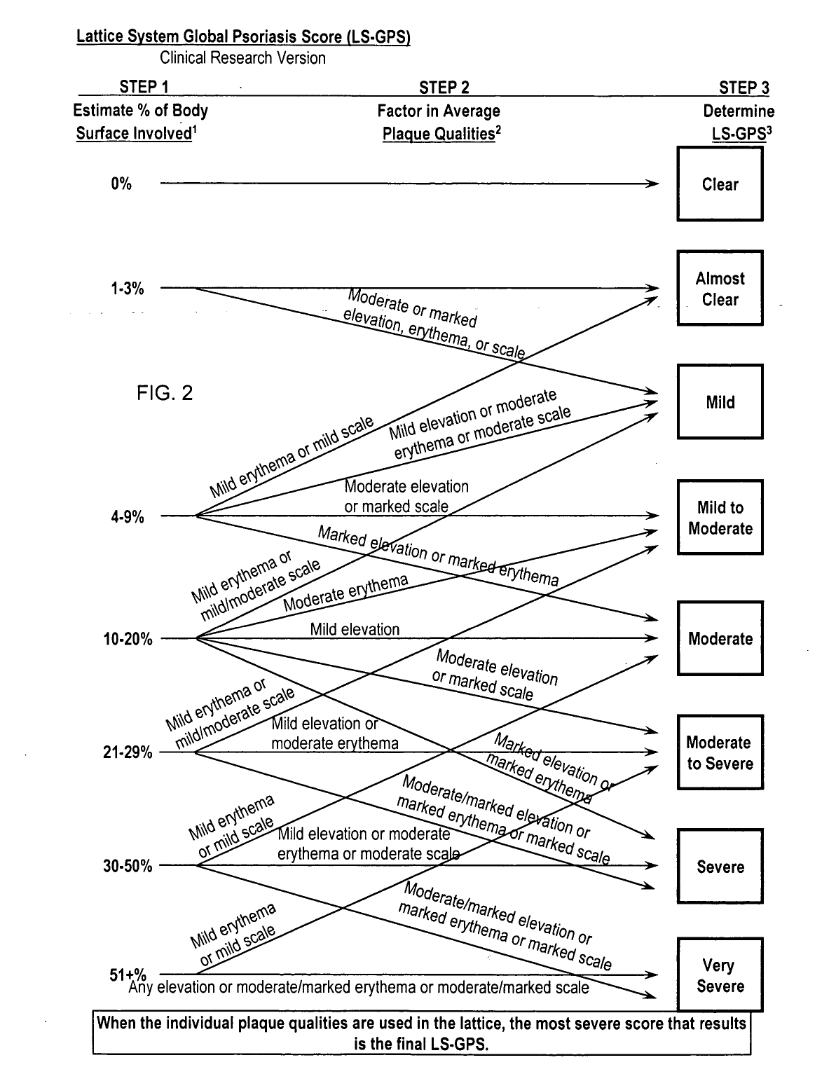

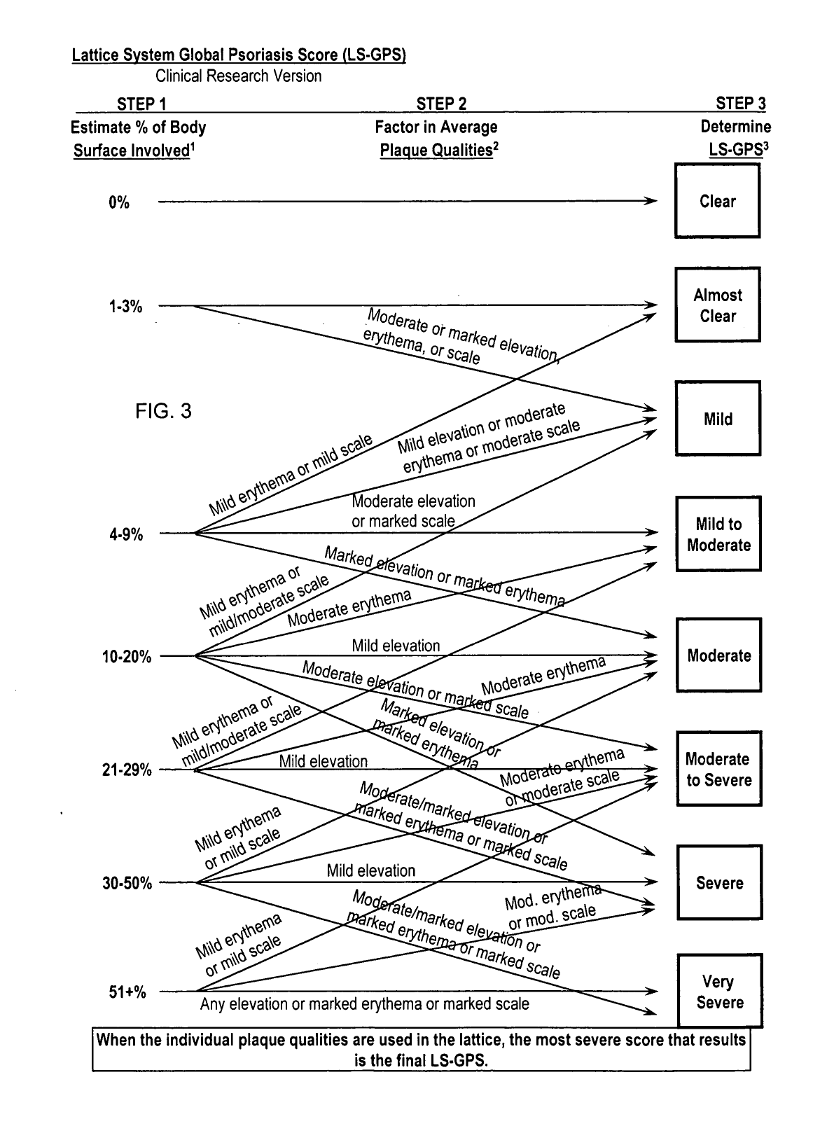

The LS-GPS system may be implemented through hardware, software, or a combination thereof. The patient assessments may be accomplished by manual inspection or with the aid of instrumentation, such as a chromameter for erythema and/or an ultrasound for elevation, or other types of measurement devices. The assessments may be recorded on a computerized input form similar to FIG. 1. Once completed, a computer may identify the severity of psoriasis for the patient (i.e., results) based on the information provided in the input form using logic similar to what is depicted in FIGS. 2 or 3. The logic may be implemented in software, hardware, or a combination thereof. The computerized input form and the results may be presented as Web pages that can be displayed via a Web browser. The logic may be embedded within Web pages or accessible via Web pages. Thus, the LS-GPS system may be implemented via the Internet.

Alternatively, the LS-GPS system may be implemented through manual techniques in combination with charts or forms resembling FIG. 1 and FIGS. 2 or 3. For example, the global score may be determined using a graphic system, such as shown in FIGS. 2 or 3, or through a software program. The global score is used to classify the severity of the patient's psoriasis into four or more classes.

In one embodiment, the system provides static methods for assessing the severity of psoriasis in a patient. The method comprises assessing the percentage of the patient's body surface that exhibits signs of psoriasis, assessing the overall elevation, erythema, and scale of the areas that exhibit signs of psoriasis, and obtaining a global score of the patient's psoriasis on the basis of the percentage of the patient's body surface that exhibits signs of psoriasis adjusted by the extent of elevation, erythema and scale of the involved areas. The weight given to elevation in obtaining the global score is greater than the weight given to erythema or scale. In certain cases, the weight given erythema in obtaining the global score is greater than the weight given scale. The global score is used to classify the severity of the patient's psoriasis into four or more classes. In various embodiments, the severity of the patient's psoriasis is classified into 4-10 classes. Good results have been obtained by classifying the severity of the patient's psoriasis into eight classes.

The percentage of the patient's body surface exhibiting signs of psoriasis is measured or estimated by a qualified person. Preferably, the amount of the body surface area that is involved with psoriasis is qualified into a plurality of increasing ranges, preferably four to ten ranges of increasing involvement. Good results have been obtained by grouping the percentage into the following seven groups: 0%, 1-3%, 4-9%, 10-20%, 21-29%, 30-50% and 51-100%.

With reference to FIG. 1, in an exemplary embodiment of the LS-GPS, the severity of the overall quality of the psoriasis plaques for elevation, erythema, and scale is categorized into a plurality of categories of increasing severity with the lowest category encompassing plaques that exhibit no elevation, erythema, or scale, respectively. Preferably, elevation, erythema, and scale are categorized into two to eight categories. Good results have been achieved when elevation, erythema, and scale are categorized into four categories which in the present case are designated as none, mild, moderate, and marked, for convenience.

Preferably scale is ignored in determining the plaque elevation. In the four category system, the extent of elevation is placed in the lowest category, i.e., none, if overall there is no elevation of the psoriasis plaques above the normal skin of the patient. The extent of elevation preferably is placed in the next to lowest category, i.e., mild, if there is a slight elevation above surrounding normal skin, and the edges are typically barely palpable and not seen. The extent of elevation preferably is placed in the next to highest category, i.e., moderate, if there is a visually apparent elevation in the plaque edges, and the plaques have readily palpated edges with rounded or sloped edges. The extent of elevation preferably is placed in the highest category, i.e., marked, if there is a visually obvious elevation in the plaque edges, and the edges are easily palpated and the plaques have sharp edges that provide a discernible bump when sliding the finger from normal skin to the plaque.

In the four category embodiment, the severity of the overall quality of the psoriasis plaques for their erythema is categorized in the lowest category, i.e., none, if the plaques have normal skin color or post-inflammatory color change. Erythema preferably is placed in the next to lowest category, i.e., mild, if the plaques have pink color, including “residual hyperemia” (“end of therapy hyperemia”). Erythema preferably is placed in the next to highest category, i.e., moderate, if the plaques are a light to medium red color, and in the highest category, i.e. marked, if the plaques are a bright, full or deep red color.

In the four category embodiment, the severity of the overall quality of the psoriasis plaques for their scale is categorized in the lowest category, i.e., none, if there are no scales present in the involved areas, and, preferably, in the next to lowest category, i.e., mild, if fine scales looking like dust are present. The overall quality of the scales preferably is categorized in the next to highest category, i.e., moderate, if individual scales are discernible that are yellow to silver in color in the involved areas and in the highest category, i.e., marked, if the scales are coarse and thick, and yellow or silver in color, and the plaque has rough edges giving the impression that the scales could be lifted with a fingernail.

In alternative embodiments, the assessments may include thickness, induration or infiltration instead of elevation, or color or redness instead of erythema, and/or scaling or desquamation instead of scale. In such alternative embodiments any one of these alternative assessments or any combination thereof may be used.

Good results have been achieved employing a system that classifies the severity of the patient's psoriasis into eight classes, characterized as “Clear,” “Almost Clear,” “Mild,” “Mild to Moderate,” “Moderate,” “Moderate to Severe,” “Severe,” and “Very Severe.” One example of such an eight class method is shown in FIG. 2. In this system, labeled LS-GPS-1, patients with 0% involvement receive a final rating of “Clear.” Patients with 1-3% involvement receive a final rating of “Almost Clear,” unless they have moderate or marked elevation, erythema, or scale in which case they receive a final rating of “Mild.” Patients with 4-9% involvement receive a final rating of “Almost Clear” if they have mild erythema or mild scale, unless they have mild elevation or moderate erythema or moderate scale, in which case they receive a final rating of “Mild,” unless they have moderate elevation or marked scale, in which case they receive a final rating of “Mild-to-Moderate,” unless they have marked elevation or marked erythema, in which case they receive a final rating of “Moderate.” Patients with 10-20% involvement receive a final rating of “Mild” if they have mild erythema or mild or moderate scale, unless they have moderate erythema, in which case they receive a final rating of “Mild-to-Moderate,” unless they have mild elevation, in which case they receive a final rating of “Moderate,” unless they have moderate elevation or marked scale, in which case they receive a final rating of “Moderate-to-Severe,” unless they have marked elevation or marked erythema, in which case they receive a final rating of “Severe.” Patients with 21-29% involvement receive a final rating of “Mild-to-Moderate” if they have mild erythema or mild or moderate scale, unless they have mild elevation or moderate erythema, in which case they receive a final rating of “Moderate-to-Severe,” unless they have moderate or marked elevation or marked erythema or marked scale, in which case they receive a final rating of “Severe.” Patients with 30-50% involvement receive a final rating of “Moderate” if they have mild erythema or mild scale, unless they have mild elevation or moderate erythema or moderate scale, in which case they receive a final rating of “Severe,” unless they have moderate or marked elevation or marked erythema or marked scale, in which case they receive a final rating of “Very Severe.” Patients with over 50% involvement receive a final rating of “Moderate-to-Severe” if they have mild erythema or mild scale; otherwise they receive a final rating of “Very Severe.” When such method is used, the most severe score that results is the final rating.

Another eight class system, referred to herein as LS-GPS-2, is shown in FIG. 3. In the LS-GPS-2 system, patients with 0% involvement receive a final rating of “Clear.” Patients with 1-3% involvement receive a final rating of “Almost Clear,” unless they have moderate or marked elevation, erythema, or scale in which case they receive a final rating of “Mild.” Patients with 4-9% involvement receive a final rating of “Almost Clear” if they have mild erythema or mild scale, unless they have mild elevation or moderate erythema or moderate scale, in which case they receive a final rating of “Mild,” unless they have moderate elevation or marked scale, in which case they receive a final rating of “Mild-to-Moderate,” unless they have marked elevation or marked erythema, in which case they receive a final rating of “Moderate.” Patients with 10-20% involvement receive a final rating of “Mild” if they have mild erythema or mild or moderate scale, unless they have moderate erythema, in which case they receive a final rating of “Mild-to-Moderate,” unless they have mild elevation, in which case they receive a final rating of “Moderate,” unless they have moderate elevation or marked scale, in which case they receive a final rating of “Moderate-to-Severe,” unless they have marked elevation or marked erythema, in which case they receive a final rating of “Severe.” Patients with 21-29% involvement receive a final rating of “Mild-to-Moderate” if they have mild erythema or mild or moderate scale, unless they have moderate erythema, in which case they receive a final rating of “Moderate,” unless they have mild elevation, in which case they receive a final rating of “Moderate-to-Severe,” unless they have moderate or marked elevation or marked erythema or marked scale, in which case they receive a final rating of “Severe.” Patients with 30-50% involvement receive a final rating of “Moderate” if they have mild erythema or mild scale, unless they have moderate erythema or moderate scale, in which case they receive a final rating of “Moderate to Severe,” unless they have mild elevation, in which case they receive a final rating of “Severe,” unless they have moderate or marked elevation or marked erythema or marked scale, in which case they receive a final rating of “Very Severe.” Patients with over 50% involvement receive a final rating of “Moderate-to-Severe” if they have mild erythema or mild scale; unless they have moderate erythema or moderate scale, in which case they receive a final rating of “Severe,” unless they have any elevation or marked erythema or marked scale, in which case they receive a final rating of “Very Severe.” When such method is used, the most severe score that results is the final rating.

In additional embodiments, other assessments, referred to herein as “disabling findings,” may be used to adjust the ratings. Such disabling findings include involvement of the face, groin, palms, soles, or axilla with psoriasis. Such disabling findings are used to increase the level of severity of the overall classification. For example, one to two levels of severity may be added to the overall rating if one to two disabling findings are present or two to three levels of severity if two to five disabling findings are present. In a further embodiment, the global score is increased by two levels of severity if plaques with elevation, scale, or erythema or a combination thereof are present on the palms, soles, or groin and by three levels of severity plaques with elevation, scale, erythema, or a combination thereof are present on the soles, palms, or groin of the patient.

In additional embodiments, the method may be simplified to make it more accessible to a wider group of raters, such as might be used by health care providers in a private practice setting.

### EXAMPLES

The following example is for purposes of illustration only and is not intended to limit the scope of the invention as defined in the claims which are appended hereto. The references cited in this document are specifically incorporated herein by reference.

### Example 1

Studies were conducted to quantify the relative variation in two commonly used outcome measures (the Psoriasis Area and Severity Index (PASI), and the Psoriasis Global Assessment (PGA)), and one embodiment of the present method, the Lattice System Global Psoriasis Score (LS-GPS-1).

Psoriasis rating systems can be described as static or dynamic. Static refers to scores that are not based on previous scores but rather only on findings at the time the score is rendered. Dynamic scores relate the patient's current condition to that of a previous time, usually the baseline value. Dynamic scores typically have descriptions such as “worse than at baseline,” “better than at baseline,” and “much better than at baseline.” All three rating methods in our study are static and objective (defined as being measured by an observer).

Method

Thirty-five patients with psoriasis were recruited for the study from the outpatient department, phototherapy unit, and the Day Treatment Center of the University of Michigan Department of Dermatology. Dermatology nurses asked patients to participate with a goal to recruit a range of severity of psoriasis; patients were compensated for their time. No medications were used during the study. The patients were not participating in other research studies nor were they invited to do so as part of this project. The study was approved by the University of Michigan Health System Institutional Review Board.

Patients' ages ranged from 22 to 62 years (median 42); 66% (23/35) were men and 34% (12/35) were women. Thirty-one (89%) were Caucasian, two were African-American (6%), one was Asian (3%), and one was Hispanic (3%). Patients received numbers; their names were not used. Prior to beginning the study, each patient completed the Dermatology Life Quality Index questionnaire which asks patients to assess how much their skin condition affected their quality of life over the previous week.

The study was designed for each subject to be evaluated twice by each of 17 physicians, first in the morning, and, after a luncheon break, again in the afternoon. In the morning, patients were randomly assigned to examination rooms in the University of Michigan outpatient building and were re-randomized in the afternoon. No rooms had windows and all were identical in layout; all patients wore identical examination gowns. In each session, the investigators were randomly assigned a starting room and proceeded in a room-to-room sequence until all patients were evaluated with the three rating systems.

Investigators reported the number of psoriasis clinical trials in which they had used the PASI or PGA and were classified as experienced if they had previously been investigators in three or more such clinical trials. Nine (53%) of the investigators were experienced and eight (47%) were inexperienced. Investigators' years of practicing dermatology ranged from zero (a senior resident in dermatology) to 39 years (median 14 years for experienced investigators, 13 for inexperienced). Of the experienced investigators, two had participated in 4-10, four in 10-20, and three in greater than 20 clinical trials using the PASI or PGA. Training on the day of the evaluations consisted of a brief review of the methods of the three rating systems and general instructions on recording the data, lasting approximately 30 minutes. Of the expected number of evaluations (1,190 for each rating method), 94 percent were completed appropriately to permit comparison of the rating systems. The investigators marked case report forms for the PGA score and each of the components of the PASI and LS-GPS; the final score for each evaluation using the latter two methods was tabulated later by computerized algorithm.

Statistical Analysis

Correlations among the three measures were evaluated by Spearman's correlation coefficients; Spearman's partial coefficients, adjusted for demographic values, were used with quality of life. Analysis of variance (ANOVA) was used to assess the variability in PASI, PGA and LS-GPS within and among individual raters, and to compare experienced and inexperienced raters. Reliability was determined overall by Cronbach's alpha coefficient and by the intra-class coefficient for agreement between the two sessions. PASI was rounded to the nearest integer.

Results

Relationships of the Rating Systems to Each Other

The Spearman correlation coefficients for all investigators for PASI and PGA (0.87), PASI and LS-GPS (0.86), and PGA and LS-GPS (0.83) indicate the measures were highly correlated. (A coefficient of one would indicate complete agreement of the scores whereas zero would indicate absolutely no relationship among the scores.) These relationships were similar for both experienced and inexperienced raters for PASI and PGA (0.88 vs. 0.86 respectively), PASI and LS-GPS (0.87 vs. 0.86), and PGA and LS-GPS (0.84 vs. 0.83).

Relative Intra-Rater and Inter-Rater Variation

Table 3 summarizes the intra-rater and inter-rater variation of psoriasis assessments of PASI, PGA and LS-GPS based on an analysis of variance (ANOVA). For all investigators, low intra-rater variation occurred with the PGA (sigma=0.2) and the LS-GPS (sigma=0.5); the variation with the PASI was higher (sigma=2.5). The intra-rater variation for PASI among inexperienced investigators (sigma=3.2) was almost three times higher than that for experienced investigators (sigma=1.2). Thus, when using the PASI score to assess severity of psoriasis, experienced investigators were more consistent in evaluating patients at morning and afternoon sessions than were inexperienced investigators. Experience had a negligible effect on the PGA and LS-GPS (Table 3).

As expected, the inter-rater variation was greater than intra-rater variation for all rating systems. For all investigators, PASI had the highest variation (sigma=8.8) compared to PGA (sigma=l.2) and LS-GPS (sigma=1.7). Experience again reduced variation more for PASI than for PGA or LS-GPS. (Table 3).

Standard deviations about the mean scores for each patient indicate the degree of consistency across investigators in rating the patient's psoriasis. For PASI scores, experienced investigators had smaller standard deviations than did inexperienced investigators for 94 percent of the patients (data not shown). However, for PGA and LS-GPS, the ratings showed no trend for inexperienced investigators to be less consistent than experienced investigators.

TABLE 3
Inter-Rater and Intra-Rater Variability

| PSORIASIS | Intra-Rater Variation | Inter-Rater Variation |
|---|---|---|
| ASSESSMENT | Sigma | Sigma |
| All Raters (N = 17) | | |
| PASI | 2.5 | 8.8 |
| PGA | 0.2 | 1.2 |
| LS-GPS | 0.5 | 1.7 |
| Experienced Raters | | |
| (N = 9) | | |
| PASI | 1.2 | 8.1 |
| PGA | 0.3 | 1.2 |
| LS-GPS | 0.4 | 1.8 |
| Inexperienced Raters | | |
| (N = 8) | | |
| PASI | 3.2 | 9.6 |
| PGA | 0.3 | 1.3 |
| LS-GPS | 0.5 | 1.5 |

ANOVA test.
PASI, Psoriasis Area and Severity Index;
PGA, Physician's Global Assessment;
LS-GPS, Lattice System Global Psoriasis Score.

Intra-Rater Variation by Change of Assessments Between Sessions

Overall reliability by Cronbach's alpha coefficient was similar for all rating systems (0.9 for each). Concordance of scores between sessions using intra-class coefficients (ICC) demonstrated highest reliability for LS-GPS (ICC=0.13). Reliability was 36 percent lower for PGA (ICC=0.08) and 49 percent lower for PASI (ICC=0.07). Among the inexperienced investigators, reliability of the PASI was particularly poor (ICC=0.04), approximately two-fold and four-fold poorer than for PGA and LS-GPS, respectively.

Relationship of Severity Scores to Quality of Life

None of the rating measures was correlated with patients' current quality of life (r<0.2 for each rating method).

Discussion

In this inter-rater and intra-rater variability study, the psoriasis assessments PASI, PGA, and LS-GPS were assessed twice in 35 subjects by 17 raters yielding over 3500 observations. The PASI, PGA, and LS-GPS were highly correlated with each other (r>0.8 for all comparisons).

The variation within each investigator was highest for PASI and particularly low for the PGA and LS-GPS; the latter method had the most reproducibility from session to session. As expected, the inter-rater variation was greater for all psoriasis measures; again, the PASI showed the most variability. The experience level of investigator affected consistency and reliability in the PASI, but not the PGA or LS-GPS.

Implications for Clinical Trials of Psoriasis Therapies

The greater variability seen between (inter-rater) than within (intra-rater) investigators is of importance for clinical trial research. The objective quantification of the severity of psoriasis before and after treatment is critical to measuring the efficacy of therapy. Therefore, to judge a treatment effect, the variation in rating a patient from time to time should be low; too great a variation in the rating method could interfere with determining treatment effect. If the intra-rater variability of a rating system is low, it should be reliable for the same investigator examining a given patient.

Recognizing that variability exists among investigators, however, has practical implications for conducting clinical trial research, particularly when more than one investigator may evaluate the same patient over time. Our data support the effort usually made in dermatologic trials to ensure that the same investigator conducts initial and follow-up evaluations. In instances where this is not possible, one may expect more “noise” (i.e., variability) in the clinical trial data.

Additional Embodiment

Sometimes a drug company is evaluating a treatment that is applied directly to the skin (e.g., a cream; this may be called a “topical treatment”). Often, especially in the early testing of such a cream, it is not applied to the entire body, but is just applied to one or a few areas (i.e., plaques) of psoriasis.

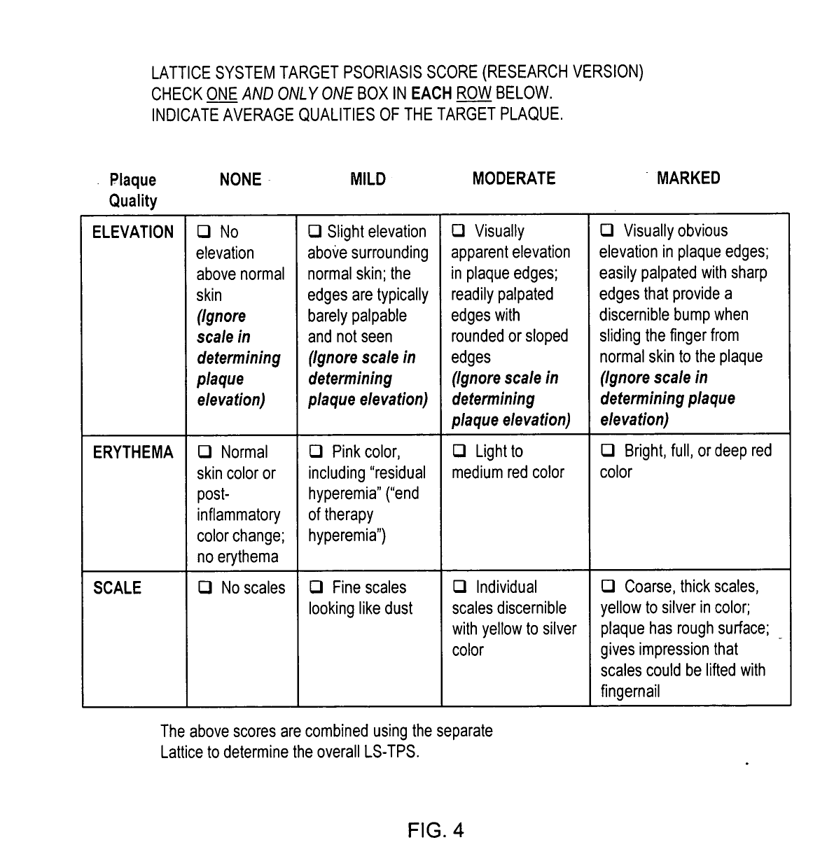

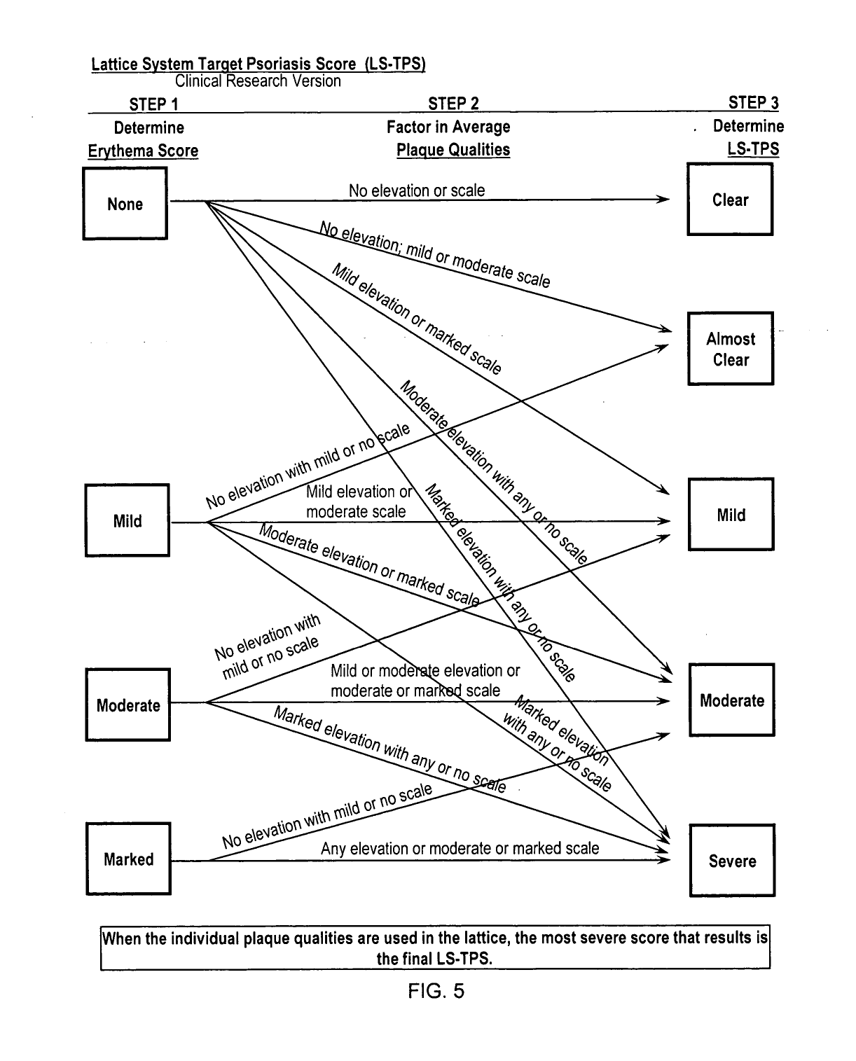

With reference to FIGS. 4 and 5, an embodiment of a lattice system for evaluating specific lesions (target plaques) of psoriasis, may be referred to as Lattice System Target Psoriasis Score (LS-TPS). This lattice concept is similar to those of FIGS. 1-3, but there is no evaluation of the “percent of the body surface that is covered” in the LS-TPS. In general, the rater still evaluates the degree of erythema (redness), the elevation of the target plaque over surrounding skin, and the amount of scale on the plaque. However, the first step is based on the erythema score, rather than the percent of body surface involved. The LS-TPS system takes the erythema, elevation, and scale together to get the final rating of clear to severe. Like the embodiments of the LS-GPS, the elevation is given more weight than the erythema or scale to reach the final score in the LS-TPS. In certain cases, erythema may be given more weight than scale in obtaining the total score. The LS-TPS system and method may be applied to an individual target area or to multiple target areas on the patient.

With reference to FIG. 4, in an exemplary embodiment of the LS-TPS, the severity of the overall quality of the psoriasis plaques for elevation, erythema, and scale is categorized into a plurality of categories of increasing severity with the lowest category encompassing plaques that exhibit no elevation, erythema, or scale, respectively. Preferably, elevation, erythema, and scale are categorized into two to eight categories. Good results have been achieved when elevation, erythema, and scale are categorized into four categories which in the present case are designated as none, mild, moderate, and marked, for convenience.

Preferably scale is ignored in determining the plaque elevation. In the four category system, the extent of elevation is placed in the lowest category, i.e., none, if overall there is no elevation of the psoriasis plaques above the normal skin of the patient. The extent of elevation preferably is placed in the next to lowest category, i.e., mild, if there is a slight elevation above surrounding normal skin, and the edges are typically barely palpable and not seen. The extent of elevation preferably is placed in the next to highest category, i.e., moderate, if there is a visually apparent elevation in the plaque edges, and the plaques have readily palpated edges with rounded or sloped edges. The extent of elevation preferably is placed in the highest category, i.e., marked, if there is a visually obvious elevation in the plaque edges, and the edges are easily palpated and the plaques have sharp edges that provide a discernible bump when sliding the finger from normal skin to the plaque.

In the four category embodiment, the severity of the overall quality of the psoriasis plaques for their erythema is categorized in the lowest category, i.e., none, if the plaques have normal skin color or post-inflammatory color change. Erythema preferably is placed in the next to lowest category, i.e., mild, if the plaques have pink color, including “residual hyperemia” (“end of therapy hyperemia”). Erythema preferably is placed in the next to highest category, i.e., moderate, if the plaques are a light to medium red color, and in the highest category, i.e. marked, if the plaques are a bright, full, or deep red color.

In the four category embodiment, the severity of the overall quality of the psoriasis plaques for their scale is categorized in the lowest category, i.e., none, if there are no scales present in the involved areas, and, preferably, in the next to lowest category, i.e., mild, if fine scales looking like dust are present. The overall quality of the scales preferably is categorized in the next to highest category, i.e., moderate, if individual scales are discernible that are yellow to silver in color in the involved areas and in the highest category, i.e., marked, if the scales are coarse and thick, and yellow or silver in color, and the plaque has rough edges giving the impression that the scales could be lifted with a fingernail.

In alternative embodiments, the LS-TPS assessment may include thickness, induration or infiltration instead of elevation, or color or redness instead of erythema, and/or scaling or desquamation instead of scale. In such alternative embodiments any one of these alternative assessments or any combination thereof may be used.

Good results may be achieved employing a system that classifies the severity of the patient's psoriasis for a target area into five classes, characterized as “Clear,” “Almost Clear,” “Mild,” “Moderate,” and “Severe.” One example of such a five class method is shown in FIG. 5. When the individual plaque qualities are used in the lattice, the most severe score that results is the final LS-TPS.

In the LS-TPS system, patients with target area(s) having an erythema score of “None” receive a final rating of “Clear” for the corresponding target area(s), unless the corresponding target area(s) have an elevation score of “Mild,” “Moderate,” or “Marked” or a scale score of “Mild,” “Moderate,” or “Marked.” With the erythema score of “None,” if the corresponding target area(s) have an elevation score of “None” and a scale score of “Mild” or “Moderate,” the patient receives a final rating of “Almost Clear” for the corresponding target area(s). With the erythema score of “None,” if the corresponding target area(s) have an elevation score of “Mild” or a scale score of “Marked,” the patient receives a final rating of “Mild” for the corresponding target area(s). With the erythema score of “None,” if the corresponding target area(s) have an elevation score of “Moderate” and a scale score of “None,” “Mild,” “Moderate, or “Marked,” the patient receives a final rating of “Moderate” for the corresponding target area(s). With the erythema score of “None,” if the corresponding target area(s) have an elevation score of “Marked” and a scale score of“None,” “Mild,” “Moderate,” or “Marked,” the patient receives a final rating of “Severe” for the corresponding target area(s).

In the LS-TPS system, patients with target area(s) having an erythema score of “Mild” receive a final rating of “Almost Clear” for the corresponding target area(s), unless the corresponding target area(s) have an elevation score of “Mild,” “Moderate,” or “Marked” or a scale score of “Moderate” or “Marked.” With the erythema score of “Mild,” if the corresponding target area(s) have an elevation score of “Mild” or a scale score of “Moderate,” the patient receives a final rating of “Mild” for the corresponding target area(s). With the erythema score of “Mild,” if the corresponding target area(s) have an elevation score of “Moderate” or a scale score of “Marked,” the patient receives a final rating of “Moderate” for the corresponding target area(s). With the erythema score of “Mild,” if the corresponding target area(s) have an elevation score of “Marked” and a scale score of “None,” “Mild,” “Moderate,” or “Marked,” the patient receives a final rating of “Severe” for the corresponding target area(s).

In the LS-TPS system, patients with target area(s) having an erythema score of “Moderate” receive a final rating of “Mild” for the corresponding target area(s), unless the corresponding target area(s) have an elevation score of “Mild,” “Moderate,” or “Marked” or a 570 scale score of “Moderate” or “Marked.” With the erythema score of “Moderate,” if the corresponding target area(s) have an elevation score of “Mild” or “Moderate” or a scale score of “Moderate” or “Marked,” the patient receives a final rating of “Moderate” for the corresponding target area(s). With the erythema score of “Moderate,” if the corresponding target area(s) have an elevation score of “Marked” and a scale score of “None,” “Mild,” “Moderate,” or “Marked,” the patient receives a final rating of “Severe” for the corresponding target area(s).

In the LS-TPS system, patients with target area(s) having an erythema score of “Marked” receive a final rating of “Moderate” for the corresponding target area(s), unless the corresponding target area(s) have an elevation score of “Mild,” “Moderate,” or “Marked” or a scale score of “Moderate” or “Marked.” With the erythema score of “Marked,” if the corresponding target area(s) have an elevation score of “Mild,” “Moderate,” or “Marked” or a scale score of “Moderate” or “Marked,” the patient receives a final rating of “Severe” for the corresponding target area(s).

The LS-TPS system may be implemented through hardware, software, or a combination thereof. The patient assessments may be accomplished by manual inspection or with the aid of instrumentation, such as a chromameter for erythema and/or an ultrasound for elevation, or other types of measurement devices. The assessments may be recorded on a computerized input form similar to FIG. 4. Once completed, a computer may identify the severity of psoriasis for the patient (i.e., results) based on the information provided in the input form using logic similar to what is depicted in FIG. 5. The logic may be implemented in software, hardware, or a combination thereof. The computerized input form and the results may be presented as Web pages that can be displayed via a Web browser. The logic may be embedded within Web pages or accessible via Web pages. Thus, the LS-GPS system may be implemented via the Internet.

Alternatively, the LS-GPS system may be implemented through manual techniques in combination with charts or forms resembling FIGS. 4 and 5. For example, the target score may be determined using a graphic system, such as shown in FIG. 5, or through a software program. The target score is used to classify the severity of the patient's psoriasis into four or more classes.

While the present invention has been illustrated by the description of embodiments thereof, and while the embodiments have been described in considerable detail, it is not the intention of the applicant to restrict or in any way limit the scope of the appended claims to such detail. Additional advantages and modifications will readily appear to those skilled in the art. For example, components and component relationships can be changed without changing the substantive functions performed by the components and component relationships described herein. Therefore, the inventive concept, in its broader aspects, is not limited to the specific details, the representative apparatus, and illustrative examples shown and described. Accordingly, departures may be made from such details without departing from the spirit or scope of the applicant's general inventive concept.

### REFERENCES

1. Marks R, Barton S P, Shuttleworth D, Finlay A Y. Assessment of disease progress in psoriasis. Arch Dermatol 1989;125(2):235-40.
2. Mckenna K E, Stern R S. The outcomes movement and new measures of the severity of psoriasis. J Am Acad Dermatol 1996;34(3):534-8.
3. Ashcroft D M, Wan Po A L, Williams H C, Griffiths C E. Clinical measures of disease severity and outcome in psoriasis: a critical appraisal of their quality. Br J Dermatol 1999;141(2):185-91.
4. Gadenne A S, Bigby M. Understanding and evaluating clinical trials. J Am Acad Dermatol 1996;34:555-590.
5. Fredriksson T, Pettersson U. Severe psoriasis—oral therapy with a new retinoid. Dermatologica 1978;157(4):238-44
6. Van de Kerkhof P C. The Psoriasis Area and Severity Index and alternative approaches for the assessment of severity: persisting areas of confusion. Br J Dermatol 1997;137(4):661-2.
7. Ormerod A D, Dwyer C M, Weller R, Cox D H, Price R. A comparison of subjective and objective measures of reduction of psoriasis with the use of ultrasound, reflectance colorimetry, computerized video image analysis, and nitric oxide production. J Am Acad Dermatol 1997;37(1):51-7.
8. Ramsay B, Lawrence C M. Measurement of involved surface area in patients with psoriasis. Br J Dermatol 1991;124(6):565-70.
9. Tiling-Grosse S, Rees J. Assessment of area of involvement in skin disease: a study using schematic figure outlines. Br J Dermatol 1993;128(1):69-74.
10. Bahmer F. The size of lesions, or point counting as a step toward the solution of the PASI problem. Arch Dermatol 1989;125(9):1282-3.
11. Lahti A, Kopola H, Harila A, Myllyla R, Hannuksela M. Assessment of skin erythema by eye, laser Doppler flow meter, spectroradiometer, two-channel erythema meter and Minolta chroma meter. Arch Dermatol Res 1993;285(5):278-82.
12. Serup J, Agner T. 72 Colorimetric quantification of erythema—a comparison of two colorimeters (Lange Micro Color and Minolta Chroma Meter CR-200) with a clinical scoring scheme and laser-Doppler flowmetry. Clin Exp Dermatol 1990;15(4):267-72.

1. A system for rating the severity of psoriasis in a patient, including:
a first logic for categorizing psoriasis plaque qualities into one of a plurality of categories of increasing severity for at least one target area of the patient based on an assessment of each psoriasis plaque quality for each corresponding target area, the psoriasis plaque qualities including elevation plaque quality, erythema plaque quality, and scale plaque quality; and
a second logic for classifying the severity of psoriasis for each target area into one of a plurality of classes of increasing severity based on the categorized psoriasis plaque quality with priority given to one or more of the plurality of categories.

2. The system of claim 1 wherein the plurality of classes include clear, almost clear, mild, moderate, and severe in increasing severity.

3. The system of claim 1 wherein the plurality of categories include none, mild, moderate, and marked in increasing severity for each psoriasis plaque quality.

4. The system of claim 3 wherein the plurality of classes include clear, almost clear, mild, moderate, and severe in increasing severity.

5. The system of claim 4 wherein the severity of psoriasis for a first target area is classified as clear when erythema plaque quality for the first target area is categorized none, elevation plaque quality for the first target area is categorized none, and scale plaque quality for the first target area is categorized none.

6. The system of claim 4 wherein the severity of psoriasis for a first target area is classified as almost clear when any of the following conditions exist: i) erythema plaque quality for the first target area is categorized none, elevation plaque quality for the first target area is categorized none, and scale plaque quality for the first target area is categorized mild or moderate, and ii) erythema plaque quality for the first target area is categorized mild, elevation plaque quality for the first target area is categorized none, and scale plaque quality for the first target area is categorized none or mild.

7. The system of claim 4 wherein the severity of psoriasis for a first target area is classified as mild when any of the following conditions exist: i) erythema plaque quality for the first target area is categorized none and elevation plaque quality for the first target area is categorized mild or scale plaque quality for the first target area is categorized marked, ii) erythema plaque quality for the first target area is categorized mild and elevation plaque quality for the first target area is categorized mild or scale plaque quality for the first target area is categorized moderate, and iii) erythema plaque quality for the first target area is categorized moderate and elevation plaque quality for the first target area is categorized none and scale plaque quality for the first target area is categorized none or mild.

8. The system of claim 4 wherein the severity of psoriasis for a first target area is classified as moderate when any of the following conditions exist: i) erythema plaque quality for the first target area is categorized none, elevation plaque quality for the first target area is categorized moderate, and scale plaque quality for the first target area is categorized none, mild, moderate, or marked, ii) erythema plaque quality for the first target area is categorized mild and elevation plaque quality for the first target area is categorized moderate or scale plaque quality for the first target area is categorized marked, iii) erythema plaque quality for the first target area is categorized moderate and elevation plaque quality for the first target area is categorized mild or moderate or scale plaque quality for the first target area is categorized moderate or marked, and iv) erythema plaque quality for the first target area is categorized marked, elevation plaque quality for the first target area is categorized none, and scale plaque quality for the first target area is categorized none or mild.

9. The system of claim 4 wherein the severity of psoriasis for a first target area is classified as severe when any of the following conditions exist: i) erythema plaque quality for the first target area is categorized none, elevation plaque quality for the first target area is categorized marked, and scale plaque quality for the first target area is categorized none, mild, moderate, or marked, ii) erythema plaque quality for the first target area is categorized mild, elevation plaque quality for the first target area is categorized marked, and scale plaque quality for the first target area is categorized none, mild, moderate, or marked, iii) erythema plaque quality for the first target area is categorized moderate, elevation plaque quality for the first target area is categorized marked, and scale plaque quality for the first target area is categorized none, mild, moderate, or marked, and iv) erythema plaque quality for the first target area is categorized marked and elevation plaque quality for the first target area is categorized mild, moderate, or marked or scale plaque quality for the first target area is categorized moderate or marked.

10. The system of claim 1, further including:
a third logic for assessing the psoriasis plaque qualities of each target area of the patient.

11. A method for rating the severity of psoriasis of a patient, including:
a) assessing psoriasis plaque qualities for at least one target area of the patient, the psoriasis plaque qualities including elevation plaque quality, erythema plaque quality, and scale plaque quality;
b) categorizing each assessed psoriasis plaque quality into one of a plurality of categories of increasing severity based on the corresponding assessed psoriasis plaque quality; and
c) classifying the severity of psoriasis for each target area into one of a plurality of classes of increasing severity based on the categorized psoriasis plaque quality with priority given to one or more of the plurality of categories.

12. The method of claim 11 wherein the plurality of classes include clear, almost clear, mild, moderate, and severe in increasing severity.

13. The method of claim 11 wherein the plurality of categories include none, mild, moderate, and marked in increasing severity for each psoriasis plaque quality.

14. The method of claim 13 wherein the plurality of classes include clear, almost clear, mild, moderate, and severe in increasing severity.

15. The method of claim 14 wherein the severity of psoriasis for a first target area is classified as clear when erythema plaque quality for the first target area is categorized none, elevation plaque quality for the first target area is categorized none, and scale plaque quality for the first target area is categorized none.

16. The method of claim 14 wherein the severity of psoriasis for a first target area is classified as almost clear when any of the following conditions exist: i) erythema plaque quality for the first target area is categorized none, elevation plaque quality for the first target area is categorized none, and scale plaque quality for the first target area is categorized mild or moderate, and ii) erythema plaque quality for the first target area is categorized mild, elevation plaque quality for the first target area is categorized none, and scale plaque quality for the first target area is categorized none or mild.

17. The method of claim 14 wherein the severity of psoriasis for a first target area is classified as mild when any of the following conditions exist: i) erythema plaque quality for the first target area is categorized none and elevation plaque quality for the first target area is categorized mild or scale plaque quality for the first target area is categorized marked, ii) erythema plaque quality for the first target area is categorized mild and elevation plaque quality for the first target area is categorized mild or scale plaque quality for the first target area is categorized moderate, and iii) erythema plaque quality for the first target area is categorized moderate and elevation plaque quality for the first target area is categorized none and scale plaque quality for the first target area is categorized none or mild.

18. The method of claim 14 wherein the severity of psoriasis for a first target area is classified as moderate when any of the following conditions exist: i) erythema plaque quality for the first target area is categorized none, elevation plaque quality for the first target area is categorized moderate, and scale plaque quality for the first target area is categorized none, mild, moderate, or marked, ii) erythema plaque quality for the first target area is categorized mild and elevation plaque quality for the first target area is categorized moderate or scale plaque quality for the first target area is categorized marked, iii) erythema plaque quality for the first target area is categorized moderate and elevation plaque quality for the first target area is categorized mild or moderate or scale plaque quality for the first target area is categorized moderate or marked, and iv) erythema plaque quality for the first target area is categorized marked, elevation plaque quality for the first target area is categorized none, and scale plaque quality for the first target area is categorized none or mild.

19. The method of claim 14 wherein the severity of psoriasis for a first target area is classified as severe when any of the following conditions exist: i) erythema plaque quality for the first target area is categorized none, elevation plaque quality for the first target area is categorized marked, and scale plaque quality for the first target area is categorized none, mild, moderate, or marked, ii) erythema plaque quality for the first target area is categorized mild, elevation plaque quality for the first target area is categorized marked, and scale plaque quality for the first target area is categorized none, mild, moderate, or marked, iii) erythema plaque quality for the first target area is categorized moderate, elevation plaque quality for the first target area is categorized marked, and scale plaque quality for the first target area is categorized none, mild, moderate, or marked, and iv) erythema plaque quality for the first target area is categorized marked and elevation plaque quality for the first target area is categorized mild, moderate, or marked or scale plaque quality for the first target area is categorized moderate or marked.

20. A system for rating the severity of psoriasis in a patient, including:
means for categorizing psoriasis plaque qualities into one of a plurality of categories of increasing severity for at least one target area of the patient based on an assessment of each psoriasis plaque quality for each corresponding target area, the psoriasis plaque qualities including elevation plaque quality, erythema plaque quality, and scale plaque quality; and
means for classifying the severity of psoriasis for each target area into one of a plurality of classes of increasing severity based on the categorized psoriasis plaque quality with priority given to one or more of the plurality of categories.

21. The system of claim 20 wherein the plurality of categories include none, mild, moderate, and marked in increasing severity for each psoriasis plaque quality.

22. The system of claim 20 wherein the plurality of classes include clear, almost clear, mild, moderate, and severe in increasing severity.

23. The system of claim 20, further including:
means for assessing the psoriasis plaque qualities of each target area of the patient.

24. The system of claim 1 wherein the classifying of the severity of psoriasis for each target area is based on the categorized psoriasis plaque quality with priority given to the categorized elevation plaque quality over other categorized plaque qualities.

25. The system of claim 1 wherein the classifying of the severity of psoriasis for each target area is based on the categorized psoriasis plaque quality with priority given to the categorized erythema plaque quality over the scale plaque quality.

26. The system of claim 1 wherein the first logic for categorizing psoriasis plaque qualities includes categorizing the elevation plaque quality for the at least one target area into a first class, a second class, a third class, or a fourth class, the elevation plaque quality being categorized in the first class when there is no elevation in the at least one target area above normal skin, the elevation plaque quality being categorized in the second class when there is slight elevation in the at least one target area above surrounding normal skin and edges of psoriasis in the at least one target area are barely palpable and not visually apparent, the elevation plaque quality being categorized in the third class when there is visually apparent elevation in edges of psoriasis in the at least one target area and the edges are readily palpated and rounded or sloped, and the elevation plaque quality being categorized in the fourth class when there is visually obvious elevation in edges of psoriasis in the at least one target area and the edges are easily palpated with sharp edges providing a discernable bump.

27. The system of claim 1 wherein the first logic for categorizing psoriasis plaque qualities includes categorizing the erythema plaque quality for the at least one target area into a first class, a second class, a third class, or a fourth class, the erythema plaque quality being categorized in the first class when there is normal skin color or post-inflammatory color change in the at least one target area, the erythema plaque quality being categorized in the second class when there is pink color in the at least one target area, the erythema plaque quality being categorized in the third class when there is light to medium red color in the at least one target area, and the erythema plaque quality being categorized in the fourth class when there is bright, full, or deep red color in the at least one target area.

28. The system of claim 1 wherein the first logic for categorizing psoriasis plaque qualities includes categorizing the scale plaque quality for the at least one target area into a first class, a second class, a third class, or a fourth class, the scale plaque quality being categorized in the first class when there are no scales in the at least one target area, the scale plaque quality being categorized in the second class when there are fine scales in the at least one target area, the scale plaque quality being categorized in the third class when there are discernable yellow to silver colored individual scales in the at least one target area, and the scale plaque quality being categorized in the fourth class when there are coarse or thick, yellow to silver colored scales in the at least one target area and the surface is rough.

29. The method of claim 11 wherein the elevation plaque quality for the at least one target area is categorized into a first class, a second class, a third class, or a fourth class, the categorizing in b) further including:
d) categorizing the elevation plaque quality in the first class when there is no elevation in the at least one target area above normal skin;
e) categorizing the elevation plaque quality in the second class when there is slight elevation in the at least one target area above surrounding normal skin and edges of psoriasis in the at least one target area are barely palpable and not visually apparent;
f) categorizing the elevation plaque quality in the third class when there is visually apparent elevation in edges of psoriasis in the at least one target area and the edges are readily palpated and rounded or sloped; and
g) categorizing the elevation plaque quality in the fourth class when there is visually obvious elevation in edges of psoriasis in the at least one target area and the edges are easily palpated with sharp edges providing a discernable bump.

30. The method of claim 11 wherein the erythema plaque quality for the at least one target area is categorized into a first class, a second class, a third class, or a fourth class, the categorizing in b) further including:
d) categorizing the erythema plaque quality in the first class when, overall, there is normal skin color or post-inflammatory color change in the at least one target area;
e) categorizing the erythema plaque quality in the second class when, overall, there is pink color in the at least one target area;
f) categorizing the erythema plaque quality in the third class when, overall, there is light to medium red color in the at least one target area; and
g) categorizing the erythema plaque quality in the fourth class when there is bright, full, or deep red color in the at least one target area.

31. The method of claim 11 wherein the scale plaque quality for the at least one target area is categorized into a first class, a second class, a third class, or a fourth class, the categorizing in b) further including:
d) categorizing the scale plaque quality in the first class when there are no scales in the at least one target area;
e) categorizing the scale plaque quality in the second class when there are fine scales in the at least one target area;
f) categorizing the scale plaque quality in the third class when there are discernable yellow to silver colored individual scales in the at least one target area; and
g) categorizing the scale plaque quality in the fourth class when there are coarse or thick, yellow to silver colored scales in the at least one target area and the surface is rough.
